(19) United States
(12) Patent Application Publication
(10) Pub. No.: US 2020/0146631 A1
(43) Pub. Date: May 14, 2020

(54) Bioelectrical Signal Measuring Apparatus Including Electric Probe Attaching to Nasal Cavity Mucosa

(71) Applicant: Korea Research Institute of Standards and Science, Daejeon (KR)

(72) Inventor: Kiwoong KIM, Daejeon (KR)

(21) Appl. No.: 16/574,573

(22) Filed: Sep. 18, 2019

(30) Foreign Application Priority Data
Nov. 8, 2018 (KR) 10-2018-0136285

(51) Int. Cl. A61B 5/00; A61B 5/04; A61B 5/0478

(52) U.S. Cl. CPC A61B 5/6852; A61B 5/6819; A61B 5/04008; A61B 5/0478; A61B 5/0538; A61B 5/0035; A61B 2562/0209; A61B 2562/06; A61B 5/0086

(57) ABSTRACT

A bioelectrical signal measuring apparatus includes a mucosa contact electrode brought into contact with a nasal cavity mucosa or an oral cavity mucosa to apply a negative pressure and to measure a bioelectrical signal, an insertion tube, having one end supporting the mucosa contact electrode, inserted into a nasal cavity and bent, and a lead wire, connected to the mucosa contact electrode and connected to an amplifier disposed outside, extending in the insertion tube.

### CROSS-REFERENCE TO RELATED APPLICATIONS

This U.S. non-provisional patent application claims priority under 35 U.S.C. § 119 to Korean Patent Application No. 10-2018-0136285, filed on Nov. 8, 2018, in the Korean Intellectual Property Office, the entire contents of which are hereby incorporated by reference.

### TECHNICAL FIELD

The present disclosure relates to a hioelectrical signal measuring apparatus and, more particularly, to a hioelectrical signal measuring apparatus, attachable to a nasal or oral cavity mucosa, for measuring a bioelectrical impedance signal and an electroencephalogram (EEG) signal.

### BACKGROUND

Conventionally, electroencephalography (EEG), magnetoencephalography (MEG), near infrared spectroscopy (NIRS), and the like, have been used as methods of measuring brain activity. EEG and MEG are used to measure electrical activities of brain nerves, and NIRS is used to measure consumption of oxygen in blood to examine energy metabolism of nerve cells. Additionally, electrical impedance measurement for measuring a structural change of brain and electrical contact means for electrical stimulation of brain have been used.

In such methods, sensors are disposed on a scalp on cranium and measure activities or states of the brain. In the case of electroencephalogram (EEG), electrodes formed of a metal or ceramic are attached to a scalp using an electroconductive adhesive or are brought into electrical contact with the scalp through a conductive gel after being fixed to a flexible hood covering a head.

Similarly to an EEG electrode, an electrode for measuring impedance is disposed on the scalp. Electric impedance tomography is a non-invasive imaging technique, which is harmless to a human body because radiation is not used, to image an electroconductivity inside a human body from bioimpedance.

In the case of near infrared ray spectroscopy (NIRS), each sensor is fixed to a flexible hood without using an adhesive to ensure optical accessibility. Near Infrared spectroscopy (NIRS) is a technique for detecting near infrared ray (wavelength: 650 to 1000 nm) projected to pass through a living body or light reflected in the living body to non-invasively detect concentrations and oxygenation of hemoglobin in brain, muscle, and other tissues.

In the case of magnetoencephalography (MEG), sensors are disposed on a separate helmet, spaced from the scalp, to measure a magnetic field generated by nerve activity. Recently, an optically-pumped atomic magnetometer is disposed on a 3D printed frame depending on a shape of an individual head, such that the MEG is measured by wearable means.

In the case of an electroencephalogram electrode and an electric impedance electrode, signal distortion or low transmittance is caused by low electroconductivity of the cranium. In the case of magnetoencephalography (MEG) or near infrared spectroscopy (NIRS), as a distance between a signal source and a sensor increases, a signal is rapidly decreases. For this reason, all of the means set forth above as prior arts are not appropriate for measuring hioelectrical signals from brain deep structures or stimulating the brain deep structures. Cerebral cortex, which is relatively easily measured from a sensor attached to a scalp, is related to a primary reaction of audiovisual and tactile sensations. However, recent researches into brain are dealing with high-order cognitive functions such as memory, emotion, and the like. To this end, it is necessary to measure a signal from a deep brain structure such as a thalamus, a hippocampus, and the like.

### SUMMARY

An aspect of the present disclosure is to provide a bioelectrical signal measuring apparatus attached to a nasal cavity mucosa to measure an electroencephalogram (EEG) signal and an electrical impedance signal.

According to an aspect of the present disclosure, a bioelectrical signal measuring apparatus includes a mucosa contact electrode brought into contact with a nasal cavity mucosa or an oral cavity mucosa to apply a negative pressure and to measure a bioelectrical signal, an insertion tube, having one end supporting the mucosa contact electrode, inserted into a nasal cavity and bent, and a lead wire, connected to the mucosa contact electrode and connected to an amplifier disposed outside, extending in the insertion tube.

In an example embodiment, the mucosa contact electrode may be in the form of a shell in which an elliptical portion and a cylindrical portion are coupled to each other, the mucosa contact electrode may include a negative pressure suction inlet having a funnel shape, and the negative pressure suction inlet may be disposed to have an inclined cross section of the elliptical portion.

In an example embodiment, the mucosa contact electrode may be a conductive metal, metal plating, conductive ceramic, or conductive silicon having low electrical contact resistance and less ionization caused by a body fluid.

In an example embodiment, the conductive metal or the metal plating may be gold, platinum, titanium, tantalum, stainless steel, platinum-iridium, or alloys thereof, the conductive ceramics may be silver chloride (AgCl), titanium nitride (TiN), surface-chloridized silver, or surface-nitrided silver, and the conductive silicon may be a silicone elastomer filled with silver-plated particles, or a silicon elastomer mixed with conductive metal particles.

In an example embodiment, the bioelectrical signal measuring apparatus may further include a valve connected to the other end of the insertion tube to control the negative pressure and to vent the negative pressure to atmosphere, a vacuum tank connected to the valve to provide a constant negative pressure, a vacuum pump exhausting the vacuum tank, and a regulator disposed between the vacuum pump and the vacuum tank to provide a constant pressure to the vacuum tank. The vacuum pump may continuously generate a negative pressure and determines the degree of vacuum in the vacuum tank using the regulator, the regulator may adjust the degree of vacuum to prevent the mucosa contact electrode from damaging the mucosa, and the valve may vent the insertion tube to an atmospheric pressure to separate the mucosa contact electrode from a surface of the mucosa.

In an example embodiment, elasticity of the insertion tube may be used or a metal core, firmed of a metal material, may be inserted into the insertion tube to fix the mucosa contact electrode.

In an example embodiment, the bioelectrical signal measuring apparatus may further include an insertion tube-nostrils coupling portion fixing the insertion tube to nostrils. The insertion tube-nostrils coupling portion may include a multilayer soft fin tube, into which the insertion tube is inserted, inserted into the nostrils, an insertion tube fixing body, disposed outside the nostrils, supporting the multilayer soft fin tube, and an insertion tube clamp, disposed on the insertion tube fixing body, fixing the insertion tube at a predetermined length. The insertion tube may penetrate through the insertion tube fixing body.

In an example embodiment, the mucosa contact electrode may be in the form of a funnel and be formed of conductive silicon, and may have flexibility.

In an example embodiment, the bioelectrical signal measuring apparatus may further include at least one auxiliary electrode to measure electroencephalography or electrical impedance.

In an example embodiment, the bioelectrical signal measuring apparatus may further include an optically-pumped atomic magnetometer or a near infrared spectroscopy sensor configured to measure a biomagnetic signal. The optically-pumped atomic magnetometer or the near infrared spectroscopy sensor may include a negative pressure fixing portion and may be fixed to the oral cavity or the nasal cavity.

According to an aspect of the present disclosure, a bioelectrical signal measuring apparatus includes a mucosa contact electrode brought into contact with a nasal cavity mucosa or an oral cavity mucosa to apply a negative pressure and to measure a bioelectrical signal, an insertion tube, having one end supporting the mucosa contact electrode, inserted into a nasal cavity and bent, and a lead wire, connected to the mucosa contact electrode and connected to an amplifier disposed outside, extending in the insertion tube. A bioelectrical signal measuring method of the bioelectrical signal measuring apparatus includes inserting a probe, including the mucosa contact electrode and the insertion tube, into a nasal cavity or an oral cavity to be fixed to a mucosa using a negative pressure, attaching an auxiliary electrode to a portion exposed outwardly of a human body, and differentially amplifying and measuring a potential difference of the mucosa contact electrode and the auxiliary electrode, or amplifying and measuring a voltage and current at one of the mucosa contact electrode and the auxiliary electrode while applying a voltage to the other electrode.

In an example embodiment, the method may further include measuring a biomagnetic signal or an extreme infrared spectroscopy signal in the oral cavity or the nasal cavity using an optically-pumped atomic magnetometer or a near infrared spectroscopy sensor fixed to the oral cavity or the nasal cavity.

### BRIEF DESCRIPTION OF THE DRAWINGS

The present disclosure will become more apparent in view of the attached drawings and accompanying detailed description. The embodiments depicted therein are provided by way of example, not by way of limitation, wherein like reference numerals refer to the same or similar elements. The drawings are not necessarily to scale, emphasis instead being placed upon illustrating aspects of the present disclosure.

FIG. 1 is a conceptual diagram illustrating a bioelectrical signal measuring apparatus according to an example embodiment of the present disclosure.

FIGS. 2A to 2C are conceptual diagrams illustrating a mucosa contact electrode and an insertion tube of the bioelectrical signal measuring apparatus FIG. 1.

FIG. 3 is a perspective view illustrating an insertion tube-nostrils coupling portion of the bioelectrical signal measuring apparatus of FIG. 1.

FIGS. 4A and 4B are conceptual diagrams illustrating a mucosa contact electrode and an insertion tube of a bioelectrical signal measuring apparatus according to another example embodiment of the present disclosure.

FIGS. 5A, 5B, and 5C are conceptual diagrams illustrating a mucosa contact electrode and an insertion tube of a bioelectrical signal measuring apparatus according to another example embodiment of the present disclosure.

FIGS. 6A and 6B illustrate a bioelectrical signal measuring apparatus and an infrared spectrometric sensor, respectively.

### DETAILED DESCRIPTION

An electroencephalography (EEG) or brain impedance measuring sensor according to an example embodiment may be disposed on a nasal cavity or nostrils to be closer to a brain deep structure to measure a large and reliable signal. In particular, since a cribriform plate of ethmoid bone, which is a perforation structure for connection of olfactory nerves, is present on a cranium between a nasal cavity and brain, electroconductivity is high.

An electroencephalography (EEG) or brain impedance measuring sensor according to an example embodiment may is in reliable contact with a surface of a mucosa, and a location thereof is stably fixed.

According to an example embodiment, a measuring sensor in a nasal cavity or an oral cavity may be brought into contact with a mucosa to be fixed thereto and may employ a sucking structure and/or a negative pressure suction structure. The measuring sensor may have flexibility.

An electroencephalography (EEG) or brain impedance measuring sensor may include at least one electrode, and may be brought into contact with a nasal cavity or an oral cavity mucosa to more precisely measure a signal or state from a deep brain structure.

In the case of magnetoencephalography (MEG) measuring a magnetic field generated from a cerebral nerve current source, the magnitude of a signal may be estimated from a distance between a sensor and a neural current source. Under the assumption that a z axis is in a direction perpendicular to a horizontal conductor surface, when a current dipole Q disposed parallel to a horizontal plane of the horizontal conductor lies in a position r_{Q}, magnitude of a z-directional magnetic field in a measurement position r is given by Equation 1, where e_{z} denotes a unit vector in the z direction, and u₀ denotes permeability.

$${B}_{2} = \frac{ {μ}_{0}}{4 π} \frac{Q \times ( r - rg ) \cdot {e}_{z}}{{\vert r - rg \vert}^{3}} \qquad Equation 1$$

As a result, it can be seen that the signal is decreased in proportion to the square of the distance.

When, for example, a measuring sensor approaches from the nasal cavity mucosa, a thaamus or a hippocampus of a deep brain structure is located within about 1.5 cm from the measuring sensor. However, when the measuring sensor approaches from the outside of a cranium, the distance is about 8 cm. As a result, when the measuring sensor is located in the nasal cavity mucosa, the magnitude of the signal of the measuring sensor may be increased by about 30 times, as compared with in the case in which the measuring sensor approaches from the outside of the cranium.

A negative pressure fixing means according to an example embodiment may be used as a means for fixing a small magnetic sensor or a near infrared ray spectroscopy (NIRS) sensor such as an optical pumping atomic magnetometer for measuring a deep brain signal measurement besides an electrode for electrical contact. Conventionally, a method of fixing a small magnetic sensor by being bitten with teeth of a subject has been used. When the small magnetic sensor is bitten with teeth, a strong electromyogram (EMG) signal is generated by excitation of muscles. The EMG signal may act as noise in measurement of a brain signal. When an optically-pumped atomic magnetometer or a near infrared spectroscopy (NIRS) sensor is fixed to a hard palate mucosa or a soft palate mucosa, the optically-pumped atomic magnetometer or the near infrared spectroscopy (NIRS) sensor may measure bioelectrical signals of brain organs.

Embodiments of the present disclosure will now be described below more fully with reference to accompanying drawings. The present disclosure may, however, be embodied in different forms and should not be constructed as limited to the embodiments set forth herein. Rather, these embodiments are provided so that this disclosure will be thorough and complete, and will fully convey the scope of the present invention to those skilled in the art.

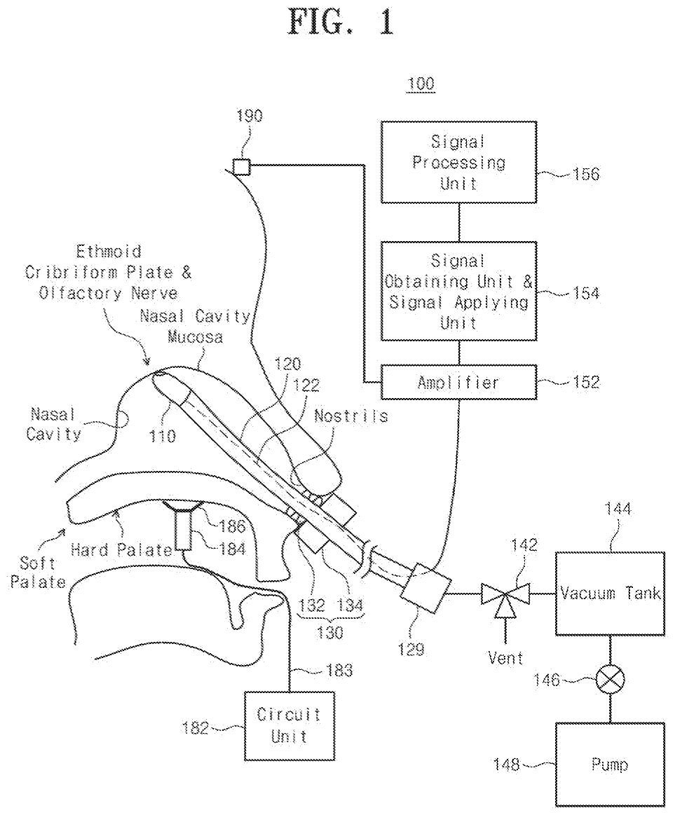

FIG. 1 is a conceptual diagram illustrating a bioelectrical signal measuring apparatus according to an example embodiment of the present disclosure.

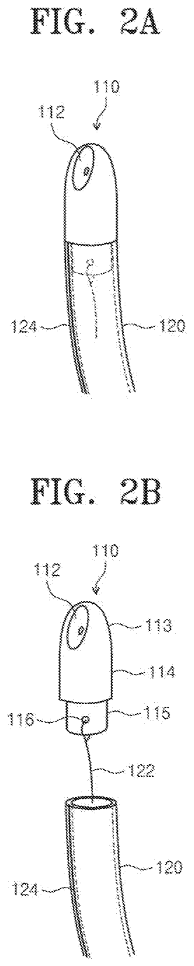

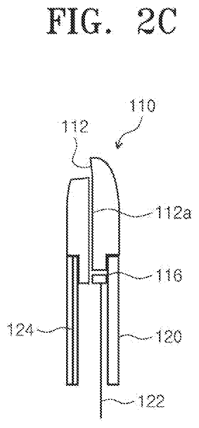

FIGS. 2A to 2C are conceptual diagrams illustrating a mucosa contact electrode and an insertion tube of the bioelectrical signal measuring apparatus of FIG. 1.

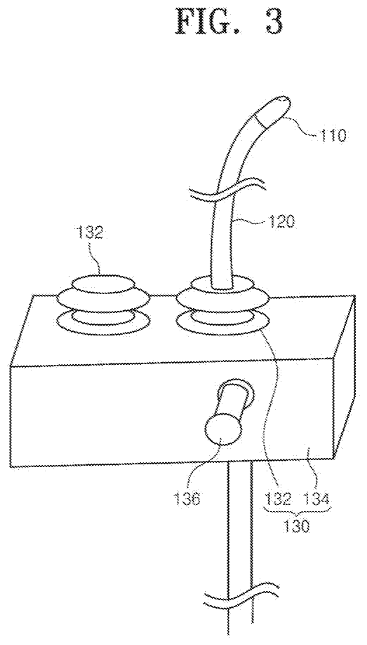

FIG. 3 is a perspective view illustrating an insertion tube-nostrils coupling portion of the bioelectrical signal measuring apparatus of FIG. 1.

Referring to FIGS. 1 to 3, a bioelectrical signal measuring apparatus 100 includes a mucosa contact electrode 110 brought into contact with a nasal cavity mucosa or an oral cavity mucosa to apply a negative pressure and to measure a bioelectrical signal, an insertion tube 120, having one end supporting the mucosa contact electrode 110, inserted into a nasal cavity and bent, and a lead wire 122, connected to the mucosa contact electrode 110 and connected to an amplifier 152 disposed outside, extending in the insertion tube 120.

The mucosa contact electrode 110 may be formed of a conductive material or may be coated with a conductive material to serves as an electrode. The mucosa contact electrode 110 may have low electrical contact resistance and less ionization caused by a body fluid. The conductive material may be a conductive metal, metal plating, conductive ceramic, or conductive silicon. The conductive metal or the metal plating may be gold, platinum, titanium, tantalum, tantalum, stainless steel, platinum-iridium, or alloys thereof. The conductive ceramics may be silver chloride (AgCl), titanium nitride (TiN), surface-chloridized silver, or surface-nitrided silver. The conductive silicon may be a silicone elastomer filled with silver-plated particles, or a silicone elastomer mixed with conductive metal particles.

The mucosa contact electrode 110 may be in the form of a shell in which an elliptical portion 113 and a cylindrical portion 114 are coupled to each other. The mucosa contact electrode 110 may include a coupling portion 115 continuously connected to the cylindrical portion 114. The coupling portion 115 may have a diameter smaller than a diameter of the cylindrical portion 114. Accordingly, when the insertion tube 120 is inserted into an external circumferential surface of the coupling portion 115, the insertion tube and the cylindrical portion may be coupled with each other without a step. The coupling portion 115 may include a connection terminal 116 for electrical connection to the lead wire 122.

The mucosa contact electrode 110 may include a negative pressure suction inlet 112 having a depressed conic shape. The negative pressure suction inlet 112 may be disposed to have an end surface inclined with respect to the elliptical portion 113. The negative pressure suction inlet 112 may be brought into contact with the mucosa such that a negative pressure is provided to stably attach the mucosa contact electrode 110 to the mucosa. The mucosa contact electrode 110 may include a through-hole 112a for externally providing a negative pressure, and the through-hole 112a may communicate with the negative pressure suction inlet 112 to provide a negative pressure to the mucosa. The through-hole 112amay extend along a central axis of the mucosa contact electrode 110 and may communicate with the insertion tube 120.

A surface of the mucosa contact electrode 110 may be applied with a local anesthetic to inhibit pain, itching, or sneezing. Alternatively, the local anesthetic may be injected into a mucosa through an additional anesthetic inflow path and the negative pressure suction inlet 112.

The insertion tube 120 may be a flexible tube having flexibility to be inserted into the nostrils. The insertion tube 120 may use flexibility of the insertion tube to fix the mucosa contact electrode 110 in the nasal cavity or the oral cavity. Alternatively, the insertion tube 120 may include a metal core 124 formed of a metal to be bent. The metal core 124 may extend alone an internal surface of the insertion tube or may be embedded in the insertion tube. The metal core 124 may have flexibility and may be bent by an external force. The insertion tube 120 may be connected to the mucosa contact electrode 110 on one end thereof and to a valve 142 or an electrical connection portion 129 at the other end thereof. The insertion tube 120 may include an electrical connection portion 129 extracting the lead wire 122 outwardly.

The lead wire 122 may be electrically connected to a connection terminal 116 of the coupling portion 115 of the mucosa contact electrode 110. The lead wire 122 may be a copper lead wire.

An insertion tube-nostrils coupling portion 130 may fix the insertion tube 120 to the nostrils. The insertion tube-nostrils coupling portion 130 includes a multilayer soft fin tube 132, into which the insertion tube 120 is inserted, inserted into the nostrils, an insertion tube fixing body 134, disposed outside the nostrils, supporting the multilayer soft fin tube, and an insertion tube clamp 136, disposed on the insertion tube fixing body 134, fixing the insertion tube 120 at a predetermined length. The insertion tube 120 may penetrate through the insertion tube fixing body 134.

A pair of multilayer soft fin tubes 132 may be disposed on one surface of the insertion tube fixing body 134 at regular intervals. The multilayer soft fin tube 132 may be formed of a soft material such as silicone to inhibit pain of a human body. The multilayer soft fin tube 132 may be inserted into nostrils of a human body. The insertion tube 120 may be inserted into the multilayer soft fin tube 132 to extend thereto. The insertion tube 120 may penetrate through the insertion tube fixing body 134 to extend therethrough. A length, at which the insertion tube 120 is inserted into the nostrils, may be adjusted through the insertion tube clamp 136. The insert tube clamp 136 may fix the insertion tube 120 using a screw-like structure or a variety of clamping structures.

A valve 142 may connected to the other end of the insertion tube 120, and may control the negative pressure and vent the negative pressure to atmosphere. The valve 142 may be a three-way valve. The valve 142 may connect the insertion tube 120 to a vacuum tank 144 in a negative pressure mode and may vent the insertion tube 120 at atmospheric pressure in a vent mode.

The vacuum tank 144 may be connected to the valve 142 to provide a constant negative pressure. The vacuum tank 144 may be a pressure reservoir maintained at a constant pressure at an atmospheric pressure or less. A pressure of the vacuum tank 144 may be constantly maintained at a pressure lower than atmospheric pressure (several hundreds of Torr). When the mucosa contact electrode 110 adsorbs the mucosa to generate a negative pressure, an excessive pressure difference may damage the mucosa. When the mucosa contact electrode 110 adsorbs the mucosa to generate a negative pressure, the vacuum tank 144 may control a regulator such that the excessive pressure difference is eliminated to increase the negative pressure to approximate atmospheric pressure.

The vacuum pump 144 can exhaust the vacuum tank 144. A regulator 146 may be disposed between a vacuum pump 148 and the vacuum tank 144 to provide a constant pressure to the vacuum tank 144. The regulator 146 may he opened in an initial state of the adsorption, and may be closed in a later state of the adsorption. The vacuum pump 148 may continuously generate a negative pressure and may determine the degree of vacuum in the vacuum tank 144 using the regulator 146. The regulator 146 may adjust the degree of vacuum to prevent the mucosa contact electrode from damaging the mucosa. The valve 142 may vent the insertion tube 120 to the atmospheric pressure to separate the mucosa contact electrode 110 from a surface of the mucosa.

The lead wire 122 may be connected to the amplifier 152 through the electrical connection portion 129. The amplifier 152 may amplify a bioelectrical signal. An output signal of the amplifier 152 is connected to a signal obtaining unit and signal applying unit 154.

An operation of the signal obtaining unit and signal applying unit 154 may vary depending on measurement of electrical impedance or measurement of electroencephalography (EEG). At least one auxiliary electrode 190 may be used to measure EEG or electrical impedance.

When the electroencephalogram (EEG) is measured, the mucosa contact electrode 110 may be used together with the auxiliary electrode 190. The amplifier 152 may amplify a measured potential signal of the mucosa contact electrode 110, and a reference potential may use a measured value at the auxiliary electrode 190. The amplifier 190 may differentially amplify the measured potential signal and the reference potential. The signal obtaining unit and signal applying unit 154 may receive an output signal of the amplifier 152 and convert the received signal into a digital signal, and may provide the digital signal to a signal processing unit 156. The signal processing unit 156 may analyze an EEG waveform or a power spectrum.

When the electrical impedance is measured, the mucosa contact electrode 110 may be used together with the auxiliary electrode 190. The amplifier 152 may be used to amplify a measured signal or to inject current. An applied current or applied voltage may be applied through the auxiliary electrode 190. The amplifier 152 may amplify current and/or a voltage flowing through the mucosa contact electrode 1110. The signal obtaining unit and signal applying unit 154 may receive an output signal of the amplifier and convert the received signal into a digital signal, and may provide the digital signal to the signal processing unit 156. The signal processor 156 may analyze an impedance signal or a power spectrum.

An optically-pumped magnetometer may include an optically-pumped magnetometer sensor 184, a negative pressure fixing portion 186 configured to fix an optically-pumped magnetometer sensor 184 to a nasal cavity or an oral cavity, a magnetometer circuit unit 182, and a cable 183 connecting the magnetometer circuit unit 182 and the optically-pumped magnetometer sensor 184 to each other. The optically-pumped magnetometer sensor 184 includes a semiconductor laser configured to output a laser beam, a lens configured to focus the laser beam, a linear polarizer and a circularly polarized polarizer configured to polarize the focused laser beam, a first reflection unit configured to provide the polarized laser beam to a vapor cell, a second reflection unit configured to reflect the laser beams passing through the vapor cell, and a photodiode configured to measure the laser beam reflected by the second reflection unit. The vapor cell may be heated to about 150 degrees Celsius. The optically-pumped magnetometer sensor 184 may be fixed in the nasal cavity or the oral cavity through the negative pressure fixing portion 186. The optically-pumped magnetometer sensor 184 may measure a magnetic signal from the deep brain structure.

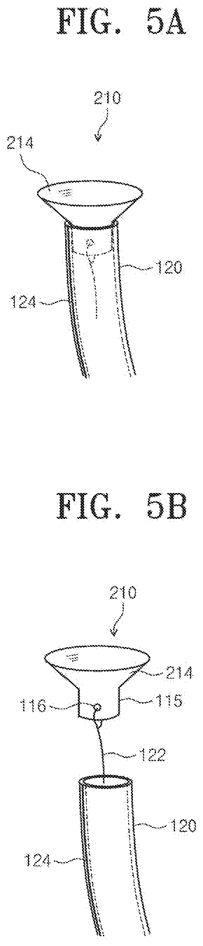

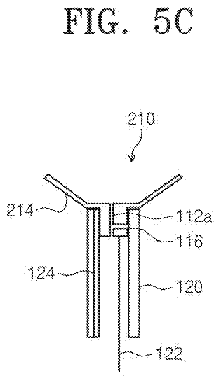

The negative pressure fixing portion 186 may be in the form of a funnel or a cone shell, similar to that of FIG. 5A, and may be formed of silicon and have flexibility. The negative pressure fixing portion 186 may be formed to have a shape of a sucker and may be fixed to the mucosa without externally applying a negative pressure. The negative pressure fix portion 186 may have a sucker structure and may be formed using silicone or the like. The negative pressure fixing portion 186 may be fixed to the oral cavity or the nasal cavity at a negative pressure without using jaw muscles to inhibit noise caused by an electromyogram (EMG) signal.

The optically-pumped magnetometer sensor 184 may be connected to the magnetometer circuit unit 182 through the cable 183. The magnetometer circuit unit 182 may amplify and filter an analog signal, and may convert the amplified and filtered signal into a digital signal. The digital signal may provide a magnetoencephalography (MEG) signal through an analysis algorithm. The MEG signal and a magnetic signal of the optically-pumped magnetometer sensor may be simultaneously analyzed to predict brain activity.

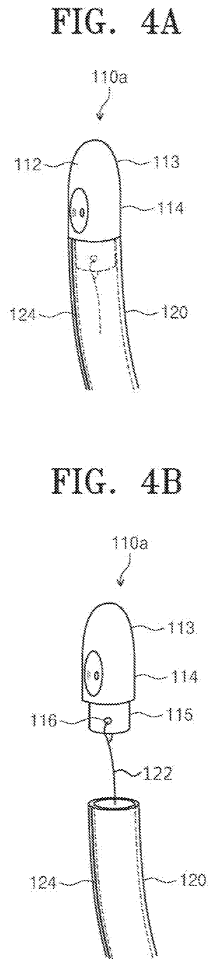

FIGS. 4A and 4B are conceptual diagrams illustrating a mucosa contact electrode and an insertion tube of a bioelectrical signal measuring apparatus according to another example embodiment of the present disclosure.

Referring to FIGS. 4A and 4B, a mucosa contact electrode 110a may be in the form of a shell in which an elliptical portion 113 and a cylindrical portion are coupled to each other. The mucosa contact electrode 110a may include a coupling portion 115 continuously connected to the cylindrical portion of an insertion tube. The coupling portion may have a diameter similar to a diameter of the cylindrical portion. Accordingly, when the insertion tube is inserted into an external circumferential surface of the coupling portion 115, the insertion tube and the cylindrical portion may be coupled to each other without a step. The coupling portion 115 may include a connection terminal for electrical connection with a lead wire.

The mucosa contact electrode 110a may include a negative pressure suction inlet 112 having a depressed conic shape. The negative pressure suction inlet 112 may be disposed on the cylindrical portion 114. The negative pressure suction inlet 112 may be brought into contact with a mucosa, such that a negative pressure may be provided to stably attach the mucosa contact electrode 110a to the mucosa. In addition, the mucosa contact electrode 110a may include a through-hole 112a for providing a negative pressure, and the through-hole 112a may communicate with the negative pressure suction inlet 112, The through-hole 112a may extend along a central axis of the mucosa contact electrode 110a.

FIGS. 5A, 5B, and 5C are conceptual diagrams illustrating a mucosa contact electrode and an insertion tube of a bioelectrical signal measuring apparatus according to another example embodiment of the present disclosure.

Referring to FIGS. 5A, 5B, and 5C, a mucosa contact electrode 210 may be in the form of a funnel or a cone shell, and may be formed of conductive silicon and have flexibility. The mucosa contact electrode 210 may include a coupling portion 115. The coupling part 115 may include a connection terminal 116 for electrical connection to a lead wire 122. The mucosa contact electrode 210 may be inserted into a nasal cavity r an oral cavity to be attached to a mucosa.

The mucosa contact electrode 210 may be formed in the shape of a sucker, or the like, to be fixed to the mucosa without externally applying a negative pressure. The mucosa contact electrode 210 may have a sucker structure, and a flexible electrode may be formed using conductive silicon or the like.

According to a modified embodiment, the mucosa contact electrode 210 may be fixed to the mucosa through an externally provided negative pressure. In this case, the mucosa contact electrode 210 may simultaneously perform functions of a negative pressure suction inlet. The mucosa contact electrode 210 may be brought into contact with a mucosa such that a negative pressure may be provided to stably attach the mucosa contact electrode 210 to the mucosa. In addition, the mucosa contact electrode 210 may have a through-hole 17a for providing a negative pressure, and the through-hole 112a may be disposed on a central axis of the funnel, The through-hole 112a may extend along a central axis of the mucosa contact electrode 210 and may communicate with an insertion tube.

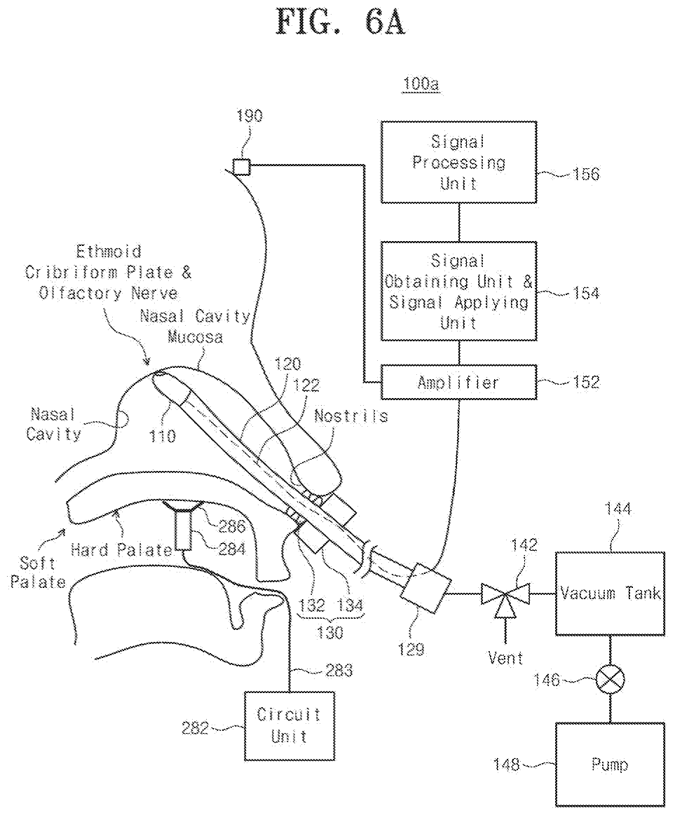

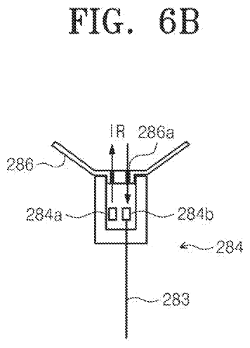

FIGS. 6A and 6B illustrate a bioelectrical signal measuring apparatus and an infrared spectrometric sensor, respectively.

Referring to FIGS. 6A and 6B, a hioelectrical signal measuring apparatus 100a includes a bioelectrical signal measuring apparatus 100 includes a mucosa contact electrode 110 brought into contact with a nasal cavity mucosa or an oral cavity mucosa to apply a negative pressure and to measure a bioelectrical signal, an insertion tube 120, having one end supporting the mucosa contact electrode 110, inserted into a nasal cavity and bent, and a lead wire 122, connected to the mucosa contact electrode 110 and connected to an amplifier 152 disposed outside, extending in the insertion tube 120.

A near infrared ray spectroscopy (NIRS) sensor 284 includes at least one light source 284a, a photodetector 284b configured to receive an infrared beam output from the light source 284a through a skin, and a negative pressure fixing portion 286 fixing a near infrared ray spectroscopy (NIRS) sensor 284 to the oral cavity. A single light source 284a may be a laser. The near infrared spectroscopy (NIRS) sensor 284 may be connected to a circuit unit 282 through a cable 283.

The negative pressure fixing portion 286 may include a through-hole 286a, through which a transparent material through which an infrared beam can pass and a laser beam is irradiated to the human body, which may receive an infrared beam traveling a human body. An EEG signal and an NIRS signal may be simultaneously analyzed to predict brain activity.

In the above-described bioelectrical signal measuring apparatus according to example embodiments, an electrode or a probe for measuring a bioelectrical signal may be stably brought into contact with an inside of a nasal cavity or an oral cavity to more precisely measure a signal in a deep brain structure which was difficult to measure in conventional brain researches. As a result, brain science research is expected to help early diagnosis of brain diseases such as dementia, Parkinson's disease, mental illness, and the like and to reduce social costs which are increasing in an aging society.

Although the present disclosure and its advantages have been described in detail, it should be understood that various changes, substitutions and alterations can be made herein without departing from the spirit and scope of the disclosure as defined by the following claims.

What is claimed is:

1. A bioelectrical signal measuring apparatus comprising:
a mucosa contact electrode brought into contact a nasal cavity mucosa or an oral cavity to apply a negative pressure and to measure a bioelectrical signal;
an insertion tube, having one end supporting the mucosa contact electrode, inserted into a nasal cavity and bent; and
a lead wire, connected to the mucosa contact electrode and connected to an amplifier disposed outside, extending in the insertion tube.

2. The bioelectrical signal measuring apparatus as set forth in claim 1, wherein the mucosa contact electrode is in the form of a shell in which an elliptical portion and a cylindrical portion are coupled to each other,
the mucosa contact electrode includes a negative pressure suction inlet having a funnel shape, and
the negative pressure suction inlet is disposed to have an inclined cross section of the elliptical portion.

3. The bioelectrical signal measuring apparatus as set forth in claim 2, wherein the mucosa contact electrode is a conductive metal, metal plating, conductive ceramic, or conductive silicon having low electrical contact resistance and less ionization caused by a body fluid.

4. The bioelectrical signal measuring apparatus as set forth in claim 3, wherein the conductive metal or the metal plating is gold, platinum, titanium, tantalum, stainless steel, platinum-iridium, or alloys thereof,
the conductive ceramics is silver chloride (AgCl), titanium nitride (TiN), surface-chloridized silver, or surface-nitrided silver, and
the conductive silicon is a silicone elastomer filled with silver-plated particles, or a silicon elastomer mixed with conductive metal particles.

5. The bioelectrical signal measuring apparatus as set forth in claim 2, further comprising:
a valve connected to the other end of the insertion tube to control the negative pressure and to vent the negative pressure to atmosphere;
a vacuum tank connected to the valve to provide a constant negative pressure;
a vacuum pump exhausting the vacuum tank; and
a regulator disposed between the vacuum pump and the vacuum tank to provide a constant pressure to the vacuum tank,
wherein the vacuum pump continuously generates a negative pressure and determines the degree of vacuum in the vacuum tank using the regulator,
the regulator adjusts the degree of vacuum to prevent the mucosa contact electrode from damaging the mucosa, and
the valve vents the insertion tube to an atmospheric pressure to separate the mucosa contact electrode from a surface of the mucosa.

6. The bioelectrical signal measuring apparatus as set forth in claim 1, wherein elasticity of the insertion tube is used or a metal core, formed of a metal material, is inserted into the insertion tube to fix the mucosa contact electrode.

7. The bioelectrical signal measuring apparatus as set forth in claim 1, further comprising:
an insertion tube-nostrils coupling portion fixing the insertion tube to nostrils,
wherein the insertion tube-nostrils coupling portion comprises:
a multilayer soft fin tube, into which the insertion tube is inserted, inserted to the nostrils;
an insertion tube fixing body, disposed outside the nostrils, supporting the multilayer soft fin tube; and
an insertion tube clamp, disposed on the insertion tube fixing body, fixing the insertion tube at a predetermined length, and
wherein the insertion tube penetrates through the insertion tube fixing body.

8. The bioelectrical signal measuring apparatus as set forth in claim 1, wherein the mucosa contact electrode is in the form of a funnel and is formed of conductive silicon, and has

9. The bioelectrical signal measuring apparatus as set forth in claim 1, further comprising:
at least one auxiliary electrode to measure electroencephalography or electrical impedance.

10. The bioelectrical signal measuring apparatus as set forth in claim 1, further comprising:
an optically-pumped atomic magnetometer or a near infrared spectroscopy sensor configured to measure a biomagnetic signal,
wherein the optically-pumped atomic magnetometer or the near infrared spectroscopy sensor includes a negative pressure fixing portion and is fixed to the oral cavity or the nasal cavity.

11. In a bioelectrical signal measuring method of a bioelectrical signal measuring apparatus including a mucosa contact electrode brought into contact with a nasal cavity mucosa or an oral cavity mucosa to apply a negative pressure and to measure a bioelectrical signal, an insertion tube, having one end supporting the mucosa contact electrode, inserted into a nasal cavity and bent, and a lead wire, connected to the mucosa contact electrode and connected to an amplifier disposed outside, extending in the insertion tube,
the bioelectrical signal measuring method comprising:
inserting a probe, including the mucosa contact electrode and the insertion tube, into a nasal cavity or an oral cavity to be fixed to a mucosa using a negative pressure;
attaching an auxiliary electrode to a portion exposed outwardly of a human body; and
differentially amplifying and measuring a potential difference of the mucosa contact electrode and the auxiliary electrode, or amplifying and measuring a voltage and current at one of the mucosa contact electrode and the auxiliary electrode while applying a voltage to the other electrode.

12. A bioelectrical signal measuring method as set forth in claim 11, further comprising:
measuring a biomagnetic signal an extreme infrared spectroscopy signal in the oral cavity or the nasal cavity using an optically-pumped atomic magnetometer or a near infrared spectroscopy sensor fixed to the oral cavity or the nasal cavity.
